(19) United States
(12) Patent Application Publication
(10) Pub. No.: US 2005/0250172 A1
(43) Pub. Date: Nov. 10, 2005

(54) Method and device for determination of hydrogen peroxide in body fluid

(75) Inventors: Osamu Nozaki, Osaka (JP); Hiroko Kawamoto, Yonago-shi (JP)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: Osamu Nozaki, Hiroko Kawamoto and Chemco Scientific Co., Ltd.

(21) Appl. No.: 10/649,471

(22) Filed: Aug. 27, 2003

(30) Foreign Application Priority Data
Aug. 28, 2002 (JP) 2002-248766

(51) Int. Cl. C12Q 1/28; G01N 21/59

(52) U.S. Cl. 435/28; 422/82.09

(57) ABSTRACT

A method for determining hydrogen peroxide and a device for use in the method excellent at determining hydrogen peroxide in body fluid in the field of a clinical examination are provided. In the method for determining hydrogen peroxide, the intensity of light emitted by reaction of immobilized horseradish peroxidase, hydrogen peroxide and imidazoles in alkaline pH is measured. The device for determining hydrogen peroxide in body fluid includes a pump for chromatography, an autosampler, a mobile phase passage, having the pump and the autosampler for body fluid, a pump for chromatography, a mobile phase passage, having the pump for a solution of imidazoles and an alkaline buffer, a flow passage, a flow cell, packed with a horseradish peroxide immobilized stationary phase, a chemiluminometer, having the flow cell, a photomultiplier, provided on the chemiluminometer in contiguity with the surface of the flow cell for the chemiluminomter. In the device, the mobile phase passages and join into the flow passage, and the flow passage is connecting to the chemiluminometer.

### BACKGROUND OF THE INVENTION

This invention relates to a method for determining hydrogen peroxide in body fluid using a novel chemiluminescence method, and to a device for use in the method.

In the conventional field of a clinical examination, it is necessary to determine hydrogen peroxide in body fluid for evaluation of oxidative stress and assay of substrates of oxidase, such as glucose, cholesterol and so on.

The determination of hydrogen peroxide by a chemiluminescence method is suitable for a clinical examination because it is simple, rapid and highly sensitive. Luminal, isoluminol, lophine, lucigenin and peroxyoxalate are generally used as light emitters for determining hydrogen peroxide by the chemiluminescence method.

However, neither of the conventional method for determining hydrogen peroxide nor conventional devices for use in the method performs rapid and effective auto-mixing of chemiluminous reagents containing hydrogen peroxide. Furthermore, the chemiluminous reaction can not be started or detected in the vicinity of a photoelectron detector, such as a photomultiplier or the like. These defects cause some problems, such as lack of repeatability of accurate determination, disposal of waste fluid of organic solvent and a decline of detecting sensitivity.

In addition, although the light emitters are essential for the conventional chemiluminescence method to determine hydrogen peroxide, they are disadvantageous and inconvenient due to difficulty in being dissolved in water, liability for having impurities and deterioration of the prepared reagents while being kept.

Furthermore, there are some problems of each of the light emitters. First of all, luminol and peroxyoxalate need to be dissolved with an organic solvent, such as acetonitrile, to prepare high concentrated solutions. Secondly, lucigenin has short duration of luminosity and causes a lot of background noises. Additionally, lophine has difficulty in being dissolved in water and low efficiency of luminosity.

### SUMMARY OF THE INVENTION

Hence, objects of the present invention are to solve the above-mentioned problems and to provide a method and a device for determining hydrogen peroxide by using a novel and quantitative chemiluninescence system which does not require the conventional light emitters in a chemiluninescence method using a flow cell for determining hydrogen peroxide.

In a method for determining hydrogen peroxide in body fluid according to the present invention, the intensity of light emitted by reaction of immobilized horseradish peroxidase, hydrogen peroxide and imidazoles in alkaline pH is measured.

Additionally, in a method for determining hydrogen peroxide in body fluid according to the present invention, the light intensity is measured in a manner as follows. Firstly, body fluid is injected into a mobile phase passage. Next, a solution of imidazoles and an alkaline buffer are injected into another mobile phase passage to be mixed with the body fluid at a place in the flow cell where a horseradish peroxidase immobilized stationary phase is packed. Consequently, light is emitted, and the light intensity is measured by a chemiluminescence detecting device.

A device for determining of hydrogen peroxide in body fluid according to the present invention includes a pump for chromatography, an autosampler, a mobile phase passage, having the pump and the autosampler for body fluid, a pump for chromatography, a mobile phase passage, having the pump for a solution of imidazoles and an alkaline buffer, a flow passage, a flow cell, packed with a horseradish peroxidase immobilized stationary phase, a chemiluminometer, having the flow cell, a photomultiplier, provided on the chemiluminometer in contiguity with the surface of the flow cell for the chemiluminometer. In the device, the mobile phase passages join into the flow passage, and the flow passage connects to the chemiluminometer.

### BRIEF DESCRIPTION OF THE DRAWING

FIG. 1 is a diagrammatic illustration of a device for determining hydrogen peroxide in body fluid according to the present invention.

### DERAILED DESCRIPTION OF THE INVENTION

Embodiments of the present invention are described below on specific, but not limited to, examples in conjunction with the accompanying drawing.

In a method for determining hydrogen peroxide in body fluid according to the present invention, the intensity of light emitted by reaction of immobilized horseradish peroxidase (hereinafter referred to as HRP), hydrogen peroxide and imidazoles in alkaline pH is measured by using a microflow-injection chemiluminescence detecting system. As a sample for the determining hydrogen peroxide, body fluid is injected from an autosampler into a mobile phase passage. A solution of imidazoles and an alkaline buffer are injected into another mobile phase passage. The body fluid, the solution of imidazoles and the alkaline buffer flow into a flow cell to be automatically mixed up at a place where a HRP immobilized stationary phase is packed. Subsequently, light is emitted in the HRP immobilized stationary phase, and the light intensity is measured by a photomultiplier of the chemiluminescence detecting system. The measured data is calculated with a data processor.

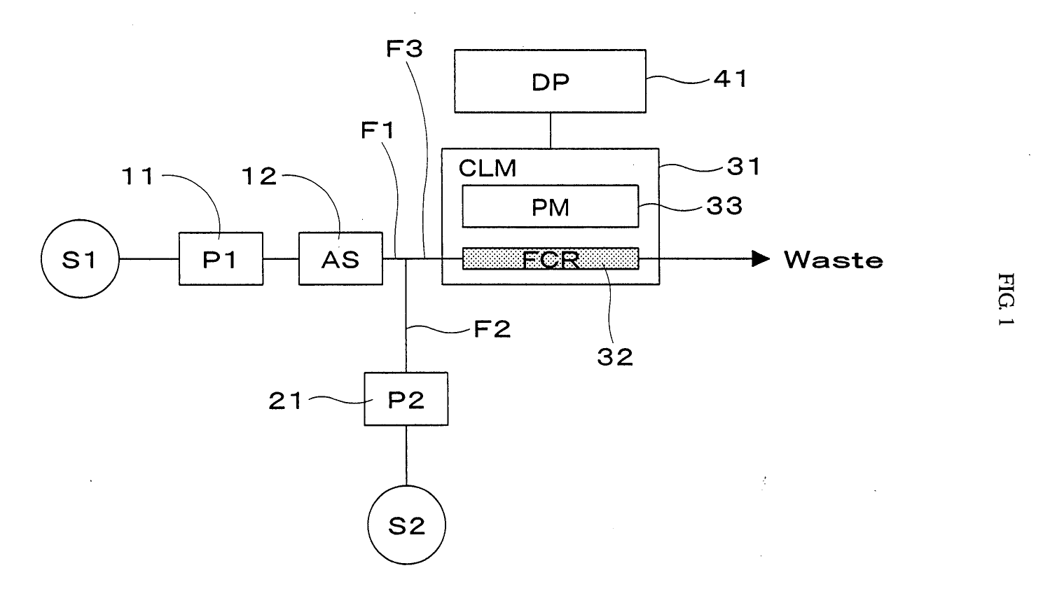

As shown in FIG. 1, a device for determining hydrogen peroxide in body fluid according to the present invention includes two pumps, 11, 21 for high performance liquid chromatography, an autosampler 12, a chemiluminometer 31 with a flow cell 32, and a data processor 41. A mobile phase passage F1, having the pump 11 and the autosampler 12 for body fluid, and a mobile phase passage F2, having the pump 21 for a solution of imidazoles and an alkaline buffer join into a flow passage F3. The joined flow passage F3 connects to the chemiluminometer 31 with the flow cell 32 where a HRP immobilized stationary phase is packed. The chemiluminometer 31 includes a photomultiplier 33 in contiguity with the surface of the flow cell 32 and electrically connects to the data processor 41.

The body fluid used in the present invention includes; for example, sweat, tears, blood, urine, sputum, lymph or the like. The amount of the necessary body fluid is approximately as little as 5-50 μL.

In the present invention, a stainless-steel tube or a Teflon® tube or the like may be used as the mobile phase passage for the body fluid, the mobile phase passage for the solution of imidazole and the alkaline buffer, and the joined flow passage of these mobile phases. The flow rates within these passages are favorably not faster than 100 μL/min.

In the present invention, as the imidazoles, imidazole, 2-methylimidazole, 4-methylimidazole, 4-methyl-5-hydroxymethylimidazole, allantoin, ethyleneurea, histidine, pyrazole or the like may be used, but not limited to them. The concentration of a solution of the imidazoles is favorably 100 mmol/L or so.

In the present invention, as the alikaline buffer, a Tricine buffer, a Tris-hydrochloric acid buffer, a boric acid buffer or the like may be used, but not limited to them. The concentration of the alkaline buffer depends on a sort of buffer. For example, when a Tricine buffer was used, it was prepared at the concentration of 50 mmol/L and the pH of 9.2.

In the present invention, as the HRP immobilized stationary phase, an amino group introduced gel, such as a chitosan gel, glass beads, a polystyrene gel, an acrylic gel, or the like may be used, but not limited to them.

In the present invention, preparation of the reagents, immobilization of the HRP and preparation of the flow cell were carried out in a manner as follows.

[Preparation of Reagents]

(1) HRP (EC1.11.1.7, 100 U/mL and over), hydrogen peroxide (H₂O₂) and imidazole (1,3-diaza-2,4-cyclopentadiene) respectively made by Wako Pure Chemical Industries, Ltd. were used.

(2) Purified water deionized by a Milli-Q system made by Nihon Millipore Co., Ltd. was used.

(3) As the stationary phase, Chitopearl beads made by Fuji Spinning Co., Ltd. and amino group introduced non-porous glass beads made by Chemco Scientific Co., Ltd. were used.

[Immobilization of HRP]

The HRP (15 mg/mL) was diluted with a phosphate buffer (0.1 mol/L, pH6.5.) and was immobilized into the stationary phase by a Nakane's method (a method for oxidizing a sugar chain).

[Preparation of a Flow Cell]

A flow cell was prepared by packing the HRP immobilized stationary phase into a Teflon® tube (the diameter of 0.96 mm) for 3 cm.

In the present invention, for example, a pump for high performance liquid chromatography made by JASCO Corporation (PU-980) can be used.

In the present invention, for example, an autosampler made by JASCO Corporation (AS-950) can be used.

In the present invention, for example, a chmiluminometer having a flow cell made by JASCO Corporation (823-CL) can be used.

In the present invention, for example, a data processor made by JASCO Corporation (LCSS-905) can be used.

The following are results of comparison of the chemiluminescence according to the present invention with the conventional luminol chemiluminescence and the conventional lophine chemiluminescence.

The reaction mechanism of the chemiluminescence according to the present invention is considered as follows. Firstly, the immobilized HRP, the hydrogen peroxide and dissoloved oxygen in the alkaline solution oxidize the imidazole to imidazole hydroperoxide. The imidazole hydroperoxide is further oxidized to imidazole dioxetane. It is deemed that the imidazole dioxetane emits light while decaying.

The intensity of light emitted by the hydrogen peroxide of the present invention was as intense as the intensity of light emitted by the hydrogen peroxide of the luminol chemiluminescence. The regression equation of the calibration curve of each of the hydrogen peroxide (1.9, 3.9, 5.6, 7.8 and 9.7 μmol/L) of the chemiluminescence according to the present invention was as follows.
Y=9860 X²+3830 X+11700
(Y: the light intensity, X: the concentration of hydrogen peroxide, μmol/L)

A range of the concentration of this hydrogen peroxide was the same as a range of the concentration of the hydrogen peroxide determined by the luminol chemiluminescence. Furthermore, the minimum detection limits of both of them were almost the same; specifically, the minimum detection limit of the chemiluminoscene according to the present invention was 0.1 μmol/L, and the minimum detection limit of the luminol chemiluminoscene was 0.2 μmol/L (in each case, S/N=2, and the amount of the hydrogen peroxide was 50 μL.)

The chemiluminescence according to the present invention showed excellent reproducibility of the determined value of the hydrogen peroxide. According to the study of within-run reproducibility of the light intensity, the coefficient of variation was 0.3% for 9.7 μmol/L of the hydrogen peroxide, 0.4% for 4.9 μmol/L of the hydrogen peroxide and 0% for 2.4 μmol/L of the hydrogen peroxide (in each case, n=5).

In the chemiluminescence according to the present invention, quality of the HRP immobilized stationary phase (made of chitosan beads and glass beads) did not affect chemiluminescence. However, the chitosan beads were superior to the glass beads for HRP stability.

The chemiluminescence according to the present invention was as sensitive as the luminol chemiluminescence and was superior for water solubility. The method for determining the hydrogen peroxide was also superior to the lophine chemiluminescene for water solubility.

As described above, the method for determining hydrogen peroxide in body fluid and the device for use in the method according to the present invention, excellent at determining hydrogen peroxide in body fluid in the field of a clinical examination, are provided.

1. A method for determining hydrogen peroxide in body fluid comprising steps of:
emitting light by reaction of immobilized horseradish peroxidase, hydrogen peroxide and imidazoles in alkaline pH; and
measuring intensity of the light.

2. A method for determining hydrogen peroxide in body fluid comprising steps of:
injecting body fluid into a first mobile phase passage;
injecting a solution of imidazoles and an alkaline buffer into a second mobile phase passage; and
mixing the body fluid with the solution of imidazoles and the alkaline buffer in a flow cell where a horseradish peroxidase immobilized stationary is packed to emit light.

3. A device for determining hydrogen peroxide in body fluid comprising:
a first mobile phase passage for body fluid, having a pump for chromatography and an autosampler;
a second mobile phase passage, having a pump for chromatography for a solution of imidazoles and an alkaline buffer;
a flow passage into which the first and second mobile phase passages join; and
a chemiluminometer to which the flow passage connects, said chemiluminometer having a flow cell where a horseradish peroxidase immobilized stationary phase is packed and a photomultiplier in contiguity with a surface of the flow cell.

4. The method for determining hydrogen peroxide in body fluid according to claim 2, wherein 5-50 μL of the body fluid is injected.

5. The method for determining hydrogen peroxide in body fluid according to claim 2, wherein a flow rate of the body fluid flowing in the mobile phase passage and a flow rate of the solution of imidazoles and an alkaline buffer flowing in the other mobile phase passage are respectively not faster than 100 μL/min.

6. The method for determining hydrogen peroxide in body fluid according to claim 4, wherein a flow rate of the body fluid flowing in the mobile phase passage and a flow rate of the solution of imidazoles and an alkaline buffer flowing in the other mobile phase passage are respectively not faster than 100 μL/min.

7. The method for determining hydrogen peroxide in body fluid according to claim 2, wherein a concentration of the solution of imidazoles is approximately 100 mmol/L.

8. The method for determining hydrogen peroxide in body fluid according to claim 4, wherein a concentration of the solution of imidazoles is approximately 100 mmol/L.

9. The method for determining hydrogen peroxide in body fluid according to claim 5, wherein a concentration of the solution of imidazoles is approximately 100 mmol/L.

10. The method for determining hydrogen peroxide in body fluid according to claim 6, wherein a concentration of the solution of imidazoles is approximately 100 mmol/L.
